(19) United States
(12) Patent Application Publication
(10) Pub. No.: US 2023/0240709 A1
(43) Pub. Date: Aug. 3, 2023

(54) MEDICAL DEVICE

(71) Applicant: TERUMO KABUSHIKI KAISHA, Tokyo (JP)

(72) Inventors: Yuichi TADA, Santa Clara, CA (US); Yoichiro KUWANO, Machida Tokyo (JP)

(21) Appl. No.: 18/161,829

(22) Filed: Jan. 30, 2023

(30) Foreign Application Priority Data
Feb. 2, 2022 (JP) 2022-014802

(51) Int. Cl. A61B 17/3207

(52) U.S. Cl. CPC A61B 17/320758; A61B 2090/065

(57) ABSTRACT

A medical device for removing an object in a body cavity, includes a rotatable drive shaft, a cutter attached to a distal end portion of the drive shaft and by which the object is cut, and one or more first sensors disposed on a distal side of the cutter, wherein each of the first sensors is configured to detect contact with the object or a biological tissue and output a signal indicating whether the first sensor is contacting the object or the biological tissue.

### CROSS-REFERENCE TO RELATED APPLICATION(S)

This application is based on and claims the benefit of priority from Japanese patent application No. 2022-014802, filed Feb. 2, 2022, the entire contents of which are incorporated herein by reference.

### BACKGROUND OF THE INVENTION

### 1. Field of the Invention

Embodiments described herein relate generally to a medical device for removing an object in a body cavity.

### 2. Description of the Related Art

Examples of treatment methods for a stenosed site caused by plaque, a thrombus, and the like in a blood vessel include a method of dilating the blood vessel by a balloon and a method of indwelling a mesh-like or coil-like stent in the blood vessel as a support for the blood vessel. However, it is difficult by these methods to treat a stenosed site that is hardened due to calcification, and a stenosed site that occurs in a bifurcated portion of the blood vessel. Examples of a method to treat such a stenosed site that is hardened include a method of cutting and removing stenotic objects such as plaque and a thrombus.

As medical devices to be used in this treatment, a medical device provided with a cutting unit that rotates to cut an object in a blood vessel has been known. The medical device provided with the cutting unit includes a drive shaft including the cutting unit in its distal end portion, and a fluid lumen through which the cut object is aspirated. The drive shaft is connected to a rotation drive source such as a motor, and the fluid lumen is connected to a fluid drive source such as a pump.

### SUMMARY OF THE INVENTION

Normally, an operator inserts a medical device into a living body, and then manually operates the medical device to perform an rotation operation such as an operation to start or stop the cutting unit. Meanwhile, a medical device control apparatus that automatically controls the operations of the medical device has been developed.

In a case where the medical device control apparatus control the medical device, it is necessary to detect that the cutting unit is appropriately in contact with a lesion area. Moreover, even in a case where the operator manually operates the medical device, if the operator can recognize whether the cutting unit is appropriately in contact with the lesion area, the operator can efficiently perform and continue the operation.

Embodiments described herein provide a medical device capable of detecting a contact state of a cutting unit with respect to a lesion area.

In one embodiment, a medical device for removing an object in a body cavity includes a rotatable drive shaft, a cutter attached to a distal end portion of the drive shaft and by which the object is cut, and one or more first sensors disposed on a distal side of the cutter, wherein each of the first sensors is configured to detect contact with the object or a biological tissue and output a signal indicating whether the first sensor is contacting the object or the biological tissue.

The medical device configured as the above can detect a contact state of the cutting unit with respect to a lesion area, and can use the information for the automatic control of the medical device or can notify an operator of the information.

### BRIEF DESCRIPTION OF THE DRAWING

FIG. 1 is a schematic view of a medical system including a medical device and a medical device control apparatus in an embodiment.

FIG. 2 is an enlarged cross-sectional view of the vicinity of a distal end portion of the medical device.

FIG. 3 is an enlarged schematic view of a first operation unit of the medical device control apparatus.

FIG. 4 is an enlarged schematic view of a second operation unit of the medical device control apparatus.

FIG. 5 is a hardware block diagram of the medical device control apparatus.

FIG. 6 is a cross-sectional view of the medical device inserted into a blood vessel in a state where a distal end portion of a distal end tube of the medical device has come into contact with a lesion area.

FIG. 7 is a cross-sectional view of the medical device inserted into the blood vessel in a state where the cutting unit has come into contact with the lesion area.

FIG. 8 is a cross-sectional view of the medical device inserted into the blood vessel in a state where an outer peripheral surface of an outer shaft of the medical device has come into contact with the blood vessel.

FIG. 9 is a schematic view of a medical device that is manually operated.

FIG. 10 is an enlarged cross-sectional view of the vicinity of a distal end portion of a medical device including a guide portion.

### DESCRIPTION OF THE EMBODIMENTS

Hereinafter, embodiment of this disclosure will be described with reference to the drawings. Note that, the size ratios in the drawings may be exaggerated for convenience of explanation, and may be different from the actual ratios in some cases. In the present specification, a side of a medical device to be inserted into a body cavity is referred to as a “distal end” or a “distal side”, and an operator-side at which the medical device is operated by the operator is referred to as a “proximal end” or a “proximal side”.

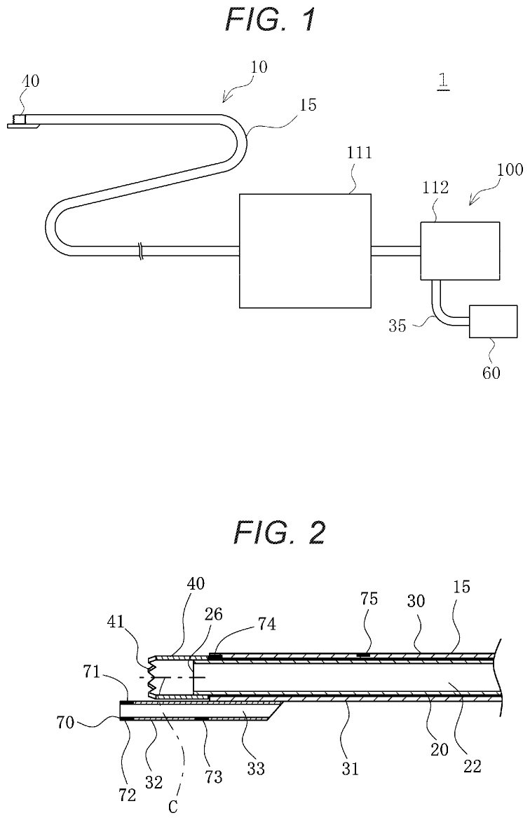

FIG. 1 is a schematic view of a medical system 1 including a medical device 10 and a medical device control apparatus 100 in one embodiment. The medical device 10 is inserted into a blood vessel, and is used in a procedure of cutting and removing a thrombus, plaque, atheroma, a calcified lesion, and the like, in acute limb ischemia and a deep venous thrombosis. Note that, an object to be removed is not necessarily limited to the thrombus, the plaque, the atheroma, and the calcified lesion, but can be any objects that may exist in a body cavity and in a body lumen.

The medical device control apparatus 100 automatically inserts the medical device 10 into a blood vessel of a patient and causes a distal end portion thereof including the cutting unit to reach the vicinity of a lesion area and cut and remove an object in the blood vessel.

As illustrated in FIGS. 1 and 2, the medical device 10 includes a shaft portion 15 including an elongated drive shaft 20 that is rotationally driven, and an outer shaft 30 that contains the drive shaft 20. A cutting unit 40 serving as a rotating body that cuts an object such as a thrombus is provided at a distal end portion of the drive shaft 20.

The drive shaft 20 transmits a rotation force to the cutting unit 40. In the drive shaft 20, a fluid lumen 22 for transporting a cut object to the proximal side is formed. The drive shaft 20 penetrates through the outer shaft 30, and has the distal end portion to which the cutting unit 40 is fixed. The drive shaft 20 includes an inlet portion 26 into which debris (e.g., a cut thrombus or the like) that is an object to be aspirated enters, at the distal end.

The drive shaft 20 is flexible, and has characteristics of allowing the power of rotation acting from the proximal side to be transmitted to the distal side. The drive shaft 20 may include one member as a whole, or may include a plurality of members. The drive shaft 20 may include a spiral-shaped slit or groove to be formed by laser processing or the like, in order to adjust the rigidity thereof depending on a site. Moreover, the distal end portion and the proximal portion of the drive shaft 20 may include different members.

As a constituent material for the drive shaft 20, for example, stainless steel, a shape memory alloy such as a nickel titanium alloy, an alloy (e.g., silver solder component) including silver, copper, zinc, and the like, an alloy (e.g., solder component) including gold, tin, and the like, cemented carbide such as tungsten carbide, polyolefin such as polyethylene and polypropylene, polyamide, polyester such as polyethylene terephthalate, a fluorinated polymer such as tetrafluoroethylene ethylene copolymer (ETFE), polyether ether ketone (PEEK), polyimide, and the like can be used suitably. Moreover, the drive shaft 20 may include a plurality of materials, or a reinforcing member such as a wire rod may be embedded.

The outer shaft 30 includes an outer shaft main body 31 that contains the drive shaft 20 so as to be rotatable, and a distal end tube 32 that is fixed to a side surface of a distal end portion of the outer shaft main body 31.

The distal end portion of the outer shaft main body 31 is positioned at a proximal side of the cutting unit 40. The outer shaft main body 31 may be in contact with or adjacent to the cutting unit 40. The outer shaft main body 31 is rotated to allow the cutting unit 40 to be oriented toward an object to be removed. Moreover, the outer shaft main body 31 may include a curved portion (not shown) that is bent at a predetermined angle in the distal end portion. The curved portion is rotated together with the outer shaft main body 31, and thus can easily cause the cutting unit 40 to contact the object to be removed.

The distal end tube 32 is fixed to an outer peripheral surface of the distal end portion of the outer shaft main body 31. The distal end tube 32 includes a distal end lumen 33 into which a guide wire can be inserted. Accordingly, the medical device 10 is a rapid exchange type device in which the distal end lumen 33 through which the guide wire is inserted is formed only in the distal end portion.

Constituent materials for the outer shaft main body 31 and the distal end tube 32 are not specially limited, and for example, stainless steel, a shape memory alloy such as a nickel titanium alloy, titanium, an alloy (e.g., silver solder component) including silver, copper, zinc, and the like, an alloy (e.g., solder component) including gold, tin, and the like, cemented carbide such as tungsten carbide, polyolefin such as polyethylene and polypropylene, polyamide, polyester such as polyethylene terephthalate, or various kinds of elastomers, a fluorinated polymer such as ETFE, PEEK, polyimide, polyacetal, and the like, can be used suitably. Moreover, the outer shaft main body 31 may include a plurality of materials, or a reinforcing member such as a wire rod may be embedded.

The cutting unit 40 is a cutter that cuts an object such as a thrombus, plaque, or calcified lesion to be small. Accordingly, “cutting” indicates that a force acts on an object to be cut, or an energy acts on such an object, thereby making the object small. An acting method of a force in the cutting, and the shape and form of the object after the cutting are not limited. The cutting unit 40 has a strength enough to cut the above-mentioned object. The cutting unit 40 is fixed to the distal end portion of the drive shaft 20. The cutting unit 40 has a cylinder shape that protrudes to the distal side from the drive shaft 20. The cutting unit 40 has a sharp blade 41 at a distal end thereof. Note that the shape of the blade 41 is not specially limited. The cutting unit 40 may include not the blade 41 but a large number of minute grinding particles. Moreover, the cutting unit 40 does not need to be a rotating body but may be a member that emits laser.

A constituent material for the cutting unit 40 preferably has a strength enough to cut a thrombus, and for example, stainless steel, titanium, diamond, ceramics, a shape memory alloy such as a nickel titanium alloy, cemented carbide such as tungsten carbide, an alloy (e.g., silver solder component) including silver, copper, zinc, and the like, high-speed steel, optical fiber, and the like can be used suitably. The constituent material for the cutting unit 40 may be resin such as polyether ether ketone (PEEK), or engineering plastic such as polyacetal.

In the medical device 10, one or more contact sensors 70 that each detect a contact state with respect to a biological tissue are provided in the vicinity of the cutting unit 40. In the present embodiment, the five contact sensors 70 of a first contact sensor 71, a second contact sensor 72, a third contact sensor 73, a fourth contact sensor 74, and a fifth contact sensor 75 are provided. The contact sensor 70 includes a pressure sensor capable of detecting the magnitude of a pressure from the outside. Moreover, the respective contact sensors 70 are connected to the proximal side of the medical device 10 by respective conductive wires that extend along the shaft portion 15, which are not illustrated. For example, those wires extend between the drive shaft 20 and the outer shaft 30 and/or outside the outer shaft 30.

The first contact sensor 71 is disposed at a distal end portion of the distal end tube 32 and on a side closer to a central axis C of the shaft portion 15. The second contact sensor 72 is disposed at the distal end portion of the distal end tube 32 and on the side farther from the central axis C of the shaft portion 15. The first contact sensor 71 and the second contact sensor 72 are provided at the distal end portion of the distal end tube 32 that extends to the distal side from the distal end of the cutting unit 40, and both are thus disposed on the distal side of the cutting unit 40.

The third contact sensor 73 is disposed at an axial direction intermediate portion of the distal end tube 32. The third contact sensor 73 is exposed to the distal end lumen 33, and can detect the pressure from a guide wire 80. The fourth contact sensor 74 is disposed on a distal end surface of the outer shaft main body 31. The fourth contact sensor 74 can detect the pressure that the distal end surface of the outer shaft main body 31 receives from the cutting unit 40. The fifth contact sensor 75 is disposed on an outer peripheral surface of the outer shaft main body 31.

The medical device control apparatus 100 includes a first operation unit 111 by which forward-and-rearward and rotary motions of the medical device 10 are controlled, and a second operation unit 112 by which rotation and aspiration of the cutting unit 40 serving as a rotating body of the medical device 10 are controlled. The first operation unit 111 and the second operation unit 112 can be connected to each other via one or more wires through which control signals from a control unit 125 (described later) are conveyed and electric power is supplied.

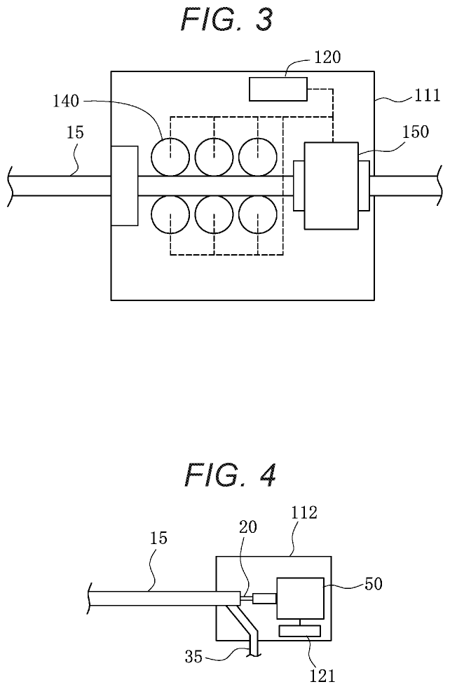

As illustrated in FIG. 3, the shaft portion 15 is inserted through the first operation unit 111, and the first operation unit 111 includes an axial direction driving unit 140 including a plurality of rollers that move the shaft portion 15 along its axial direction, and a circumferential direction driving unit 150 including a roller that surrounds the shaft portion 15 and rotates the shaft portion 15 along its circumferential direction. That is, the rotation axes of the axial direction driving unit 140 are perpendicular to the axis of the shaft portion 15, and the rotation axis of the circumferential direction driving unit 150 coincides with the axis of the shaft portion 15. The axial direction driving unit 140 and the circumferential direction driving unit 150 are connected to a device operation unit 120 serving as a drive source of these respective driving units via one or more shafts and gears. The device operation unit 120 includes, for example, an electric motor, but is not limited thereto as long as it can drive the axial direction driving unit 140 and the circumferential direction driving unit 150.

As illustrated in FIG. 4, a proximal portion of the shaft portion 15 is inserted through the second operation unit 112. In an inside of the second operation unit 112, the drive shaft 20 of the shaft portion 15 is exposed, and is connected to a rotation drive source 50. The rotation drive source 50 can include an electric motor. In the second operation unit 112, a drive operation unit 121 that performs an on-and-off operation and an operation of the rotational frequency of the rotation drive source 50 is provided. In the inside of the second operation unit 112, the shaft portion 15 is bifurcated, and a bifurcated tube 35 after the bifurcation is connected to an aspiration drive source 60. The aspiration drive source 60 can include a pump.

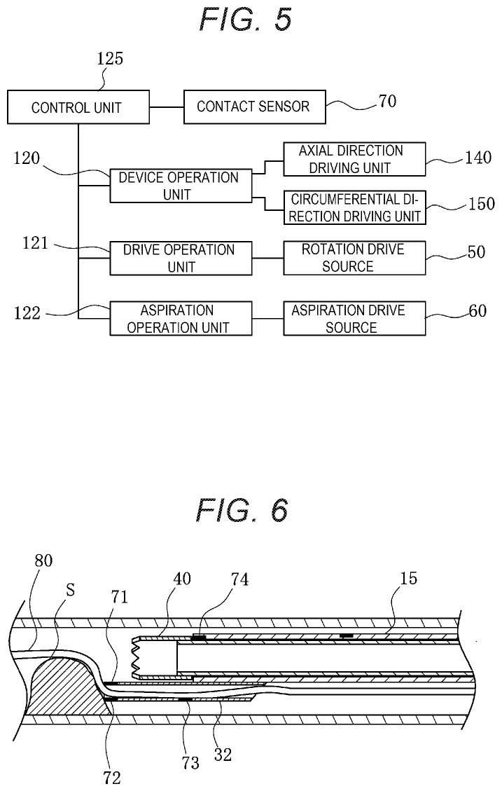

As illustrated in FIG. 5, the medical device control apparatus 100 includes a control unit 125 such as a control circuit or a controller that controls the device operation unit 120, the drive operation unit 121, and an aspiration operation unit 122. The control unit 125 is disposed in the first operation unit 111, the second operation unit 112, or an external device (not shown). The contact sensors 70 are connected to the control unit 125. As mentioned in the foregoing, the device operation unit 120 (e.g., a motor) drives the axial direction driving unit 140 and the circumferential direction driving unit 150 to control forward-and-rearward and circumferential motions of the medical device 10. Moreover, the drive operation unit 121 (e.g., a control circuit) controls the rotation drive source 50 of the medical device 10, and the aspiration operation unit 122 such as a control circuit that controls the aspiration drive source 60 of the medical device 10.

The control unit 125 includes, for example, a processor such as a central processing unit (CPU) or a micro processing unit (MPU), and a storage unit such as a memory. The control unit 125 executes a program stored in the storage unit to control the medical device 10.

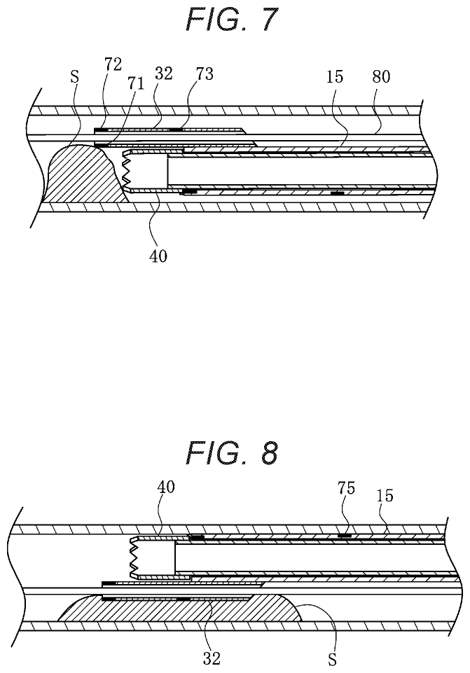

Detection of a contact state with respect to a biological tissue by the contact sensors 70 will be described. As illustrated in FIG. 6, in a case where a lesion area S is unevenly present in a part in the circumferential direction in a blood vessel, a distal end portion of the medical device 10 on a side of the distal end tube 32 in the circumferential direction comes into contact with the lesion area S in some cases. In this state, the cutting unit 40 is obstructed by the distal end tube 32, and cannot appropriately come into contact with the lesion area S. At this time, the first contact sensor 71 and the second contact sensor 72 are in contact with the hard lesion area S and the guide wire 80, and thus detect and output high pressure values, which exceed a predetermined threshold value. In this manner, in a case where both the first contact sensor 71 and the second contact sensor 72 output the high pressure values exceeding the predetermined threshold value, the control unit 125 can determine that the cutting unit 40 is not appropriately in contact with the lesion area S. In this case, as illustrated in FIG. 7, the control unit 125 controls the device operation unit 120 to rotate the cutting unit 40 to contact the lesion area S.

As illustrated in FIG. 7, in a case where the cutting unit 40 is in contact with the lesion area S, the first contact sensor 71 is in contact with the lesion area S, but the second contact sensor 72 is not in contact with the lesion area S. Accordingly, the first contact sensor 71 outputs a high pressure value exceeding the predetermined threshold value, and the second contact sensor 72 outputs a low pressure value below or equal to the predetermined threshold value. In this case, the control unit 125 can determine that the cutting unit 40 is appropriately in contact with the lesion area S. Moreover, in a case where the lesion area S is smaller, both the first contact sensor 71 and the second contact sensor 72 indicate low pressure values below or equal to the predetermined threshold value. In this case as well, the control unit 125 can determine that the cutting unit 40 is appropriately in contact with the lesion area S.

The first contact sensor 71 and the second contact sensor 72 are positioned on the distal side of the cutting unit 40, and thus can accurately detect the contact state of the cutting unit 40 with respect to the lesion area S when the medical device 10 is inserted into the blood vessel.

In a case where the guide wire 80 is largely bent by the lesion area S as shown in FIG. 6, the third contact sensor 73 can detect that the guide wire 80 has come into contact with the third contact sensor 73 in the distal end lumen 33 by detecting a high pressure value exceeding a predetermined threshold value. Accordingly, the control unit 125 can determine that the lesion area S is present on the distal end tube 32 side, and the cutting unit 40 is not appropriately in contact with the lesion area S. In this case, the control unit 125 controls the device operation unit 120 so as to rotate the cutting unit 40.

In a case where the cutting unit 40 is in contact with the lesion area S, the fourth contact sensor 74 detects a high pressure from the cutting unit 40, and thus outputs a high pressure value exceeding a predetermined threshold value. In a case where the cutting unit 40 is not in contact with a biological tissue or is in contact with a soft biological tissue such as a vascular wall, the fourth contact sensor 74 outputs a low pressure value below or equal to the predetermined threshold value. Accordingly, the control unit 125 can detect a contact state of the cutting unit 40 with respect to the lesion area S from the pressure value detected by the fourth contact sensor 74.

As illustrated in FIG. 8, the lesion area S is unevenly present in a part in the circumferential direction of a blood vessel, and the shaft portion 15 is pressed against a vascular wall on an opposite side by the lesion area S in some cases. In this state, the cutting unit 40 cannot come into contact with the lesion area S. At this time, the fifth contact sensor 75 is strongly pressed against the vascular wall, and thus outputs a high pressure value exceeding a predetermined threshold value. In this manner, in a case where the fifth contact sensor 75 outputs a high pressure value, the control unit 125 can determine that the cutting unit 40 is not appropriately in contact with the lesion area S. In this case, the control unit 125 controls the device operation unit 120 so as to move the cutting unit 40 backward in the axial direction, or to rotate the cutting unit 40.

In this manner, the contact sensors 70 are provided in the vicinity of the cutting unit 40, a contact state of the cutting unit 40 with respect to the biological tissue can be detected, and the control unit 125 can appropriately control the medical device 10 based on the information.

The contact sensors 70 are pressure sensors in the present embodiment, but sensors other than the pressure sensors may be used as long as it can detect a contact state with respect to a biological tissue. For example, the contact sensors 70 can be optical sensors. As optical sensors, for example, optical fibers can be used. By providing optical fibers that extend to the proximal portion of the medical device 10 at the respective positions of the contact sensors 70 illustrated in FIG. 2, it is possible to determine whether the cutting unit 40 is in contact with the lesion area S from the difference in colors of the biological tissue at the respective positions. Moreover, as the contact sensors 70, infrared ray sensors using the principle of optical coherence tomography (OTC) may be employed. Moreover, as the contact sensors 70, ultrasound sensors that emit ultrasound waves and detects a state of a biological tissue may be employed.

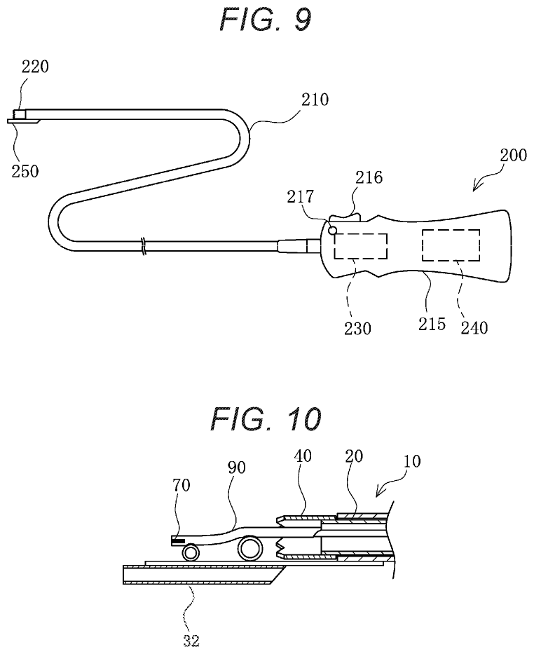

The medical device may be manually operated by an operator. As illustrated in FIG. 9, a manually operated medical device 200 in one embodiment includes a cutting unit 220 serving as a rotating body that is provided at a distal end portion of an elongated shaft portion 210, and a handle portion 215 that is gripped and operated by a hand of the operator in a proximal portion of the shaft portion 210. Inside of the handle portion 215, a rotation drive source 230, an aspiration drive source 240, and other components shown in FIG. 5 such as a control unit 125 for controlling the rotation drive source 230 and the aspiration drive source 240 are disposed. Moreover, in the handle portion 215, an operation switch 216 for operating the rotation, the aspiration, and the like of the cutting unit 220, and a notification unit 217 for notifying the operator of a contact state of the cutting unit 220 under control of the control unit 125. The notification unit 217 includes a lamp such as an LED, and can indicate a contact state of the cutting unit 220 by emitting light. Note that, the notification unit 217 may have another configuration as long as it can make a notification of the contact state, and for example, a display screen that displays a message or an icon, a speaker that generates a sound, and the like may be employed.

In the medical device 200 as well, a contact sensor 250 is provided in the vicinity of the cutting unit 220, and can detect a contact state of the cutting unit 220 with respect to a biological tissue and output the detected value to the control unit 125. The control unit 125 controls the notification unit 217 to notify the operator of the contact state based on the contact state of the cutting unit 220 detected by the contact sensor 250. Moreover, the rotation drive source 230 and the aspiration drive source 240 may be controlled based on the contact state of the cutting unit 220 detected by the contact sensor 250.

The contact sensor 70 may be provided on a member that protrudes to the distal side from the shaft portion 15. As illustrated in FIG. 10, in the medical device 10, a guide portion 90 that is inserted through the lumen of the drive shaft 20 and protrudes to the distal side from the cutting unit 40 can be provided. The guide portion 90 comes into contact with a lesion area at the distal side from the cutting unit 40, and thus limits a range in which the cutting unit 40 can come into contact with the biological tissue. In this case, the contact sensor 70 can be disposed at a distal end portion of the guide portion 90. The contact sensor 70 can be disposed on a distal end surface of the guide portion 90 or a surface thereof on the opposite side of the distal end tube 32.

As is in the foregoing, the medical device 10 includes: the elongated shaft portion 15; and the cutting unit 40 that is provided at a distal end portion of the shaft portion 15, in which the contact sensors 70 that each detect a contact state with respect to a biological tissue are provided in the vicinity of the cutting unit 40. The medical device 10 configured in this manner can detect a contact state of the cutting unit 40 with respect to a lesion area, and can output the detected contact state to the medical device control apparatus 100 for the automatic control of the medical device 10 or can notify an operator of the contact state.

Moreover, the distal end tube 32 may be interlocked with an outer peripheral surface of the shaft portion 15, and the contact sensors 70 may be attached to the distal end tube 32. Accordingly, it is possible to reliably detect a contact state of the cutting unit 40 through the contact state of the distal end tube 32 with respect to the biological tissue.

Moreover, the contact sensor 70 may be disposed at a position at a distal side from the cutting unit 40, in the distal end tube 32. Accordingly, it is possible to detect a contact state at the distal side from the cutting unit 40, and to more accurately detect a contact state of the cutting unit 40 with respect to the biological tissue.

Moreover, the contact sensors 70 may be respectively disposed on a side of the distal end tube 32 closer to the central axis C of the cutting unit 40 and on the side farther from the central axis C of the cutting unit 40. Accordingly, it is possible to more accurately detect a contact state with respect to the lesion area depending on the presence or absence of the contact in the two contact sensors 70.

Moreover, the contact sensor 70 may be disposed on the outer peripheral surface of the shaft portion 15. Accordingly, it is possible to reliably detect a contact state of the cutting unit 40 through the contact state of the outer peripheral surface of the shaft portion 15 with respect to the biological tissue.

Moreover, the shaft portion 15 may include the drive shaft 20 including the cutting unit 40 at a distal end thereof, and the outer shaft 30 through which the drive shaft 20 is inserted, the distal end portion of the outer shaft 30 may be in contact with or adjacent to the cutting unit 40, and the contact sensor 70 may be disposed in the distal end portion of the outer shaft 30. Accordingly, it is possible to reliably detect a contact state of the cutting unit 40 with respect to a hard biological tissue.

Moreover, the shaft portion 15 may include a guide portion that further extends to the distal side than the cutting unit 40, and the contact sensor 70 may be attached to the guide portion. Accordingly, it is possible to dispose the contact sensor at a position at the distal side from the cutting unit 40, and to detect a contact state with high accuracy.

Moreover, the contact sensor 70 may be a pressure sensor. The lesion area is generally hard, so that it is possible to determine whether the cutting unit 40 is in contact with the hard lesion area with high accuracy.

Moreover, the contact sensor 70 may be an optical sensor that detects light or infrared rays. Accordingly, it is possible to grasp the state of the biological tissue, and to determine a contact state with respect to the lesion area with high accuracy.

In the above-mentioned examples, the five contact sensors 70 are provided, but the number and the arrangement of the contact sensors 70 can be changed if necessary, and are not limited to those in the above-mentioned embodiments.

While certain embodiments have been described, these embodiments have been presented by way of example only, and are not intended to limit the scope of the disclosure. Indeed, the novel embodiments described herein may be embodied in a variety of other forms; furthermore, various omissions, substitutions and changes in the form of the embodiments described herein may be made without departing from the spirit of the disclosure. The accompanying claims and their equivalents are intended to cover such forms or modifications as would fall within the scope and spirit of the disclosure.

What is claimed is:

1. A medical device for removing an object in a body cavity, comprising:
a rotatable drive shaft;
a cutter attached to a distal end portion of the drive shaft and by which the object is cut; and
one or more first sensors disposed on a distal side of the cutter, wherein each of the first sensors is configured to detect contact with the object or a biological tissue and output a signal indicating whether the first sensor is contacting the object or the biological tissue.

2. The medical device according to claim 1, further comprising:
a distal end tube attached to the distal end portion of the drive shaft and through which a guide wire can pass, wherein
the first sensors are attached to the distal end tube.

3. The medical device according to claim 2, wherein the first sensors are disposed at a distal end of the distal end tube.

4. The medical device according to claim 3, wherein one of the first sensors is closest to a rotation axis of the drive shaft in a radial direction of the drive shaft, and another one of the first sensors is farthest from the rotation axis of the drive shaft in the radial direction.

5. The medical device according to claim 2, further comprising:
a second sensor disposed inside the distal end tube and configured to detect contact with the guide wire and output a signal indicating whether the second sensor is contacting the guide wire.

6. The medical device according to claim 1, further comprising:
an outer shaft surrounding the drive shaft and including a distal end portion that is in contact with or adjacent to the cutter; and
a third sensor attached to the distal end portion of the outer shaft and configured to detect contact with the object or the biological tissue and output a signal indicating whether the third sensor is contacting the object or the biological tissue.

7. The medical device according to claim 6, further comprising:
a fourth sensor disposed on a proximal side of the distal end portion of the outer shaft and configured to detect contact with the object or the biological tissue and output a signal indicating whether the fourth sensor is contacting the object or the biological tissue.

8. The medical device according to claim 1, further comprising:
a guide portion that passes through the drive shaft and protrudes from the cutter toward a distal direction, wherein
the first sensor is disposed at a distal end of the guide portion.

9. The medical device according to claim 1, wherein the first sensor is a pressure sensor.

10. The medical device according claim 1, wherein the first sensor is an optical sensor configured to detect light or infrared rays.

11. A medical system for removing an object in a body cavity, comprising:
a medical device including:
a rotatable drive shaft,
a cutter attached to a distal end portion of the drive shaft and by which the object is cut, and
one or more first sensors disposed on a distal side of the cutter, wherein each of the first sensors is configured to detect contact with the object or a biological tissue and output a signal indicating whether the first sensor is contacting the object or the biological tissue; and
a medical device control device including:
a motor configured to rotate the drive shaft,
one or more rollers configured to move the drive shaft along a rotation axis of the drive shaft and change an orientation of the drive shaft, and
a controller configured to control the rollers to move the drive shaft and/or change the orientation based on the signal output from the first sensors.

12. The medical system according to claim 11, wherein the controller is configured to control the rollers to change the orientation and then move the drive shaft toward a distal direction upon receipt of the signal indicating that the first sensor is contacting the object or the biological issue.

13. The medical system according to claim 11, wherein
the medical device further includes a distal end tube attached to the distal end portion of the drive shaft and through which a guide wire can pass, and
the first sensors are attached to the distal end tube.

14. The medical system according to claim 13, wherein the first sensors are disposed at a distal end of the distal end tube.

15. The medical system according to claim 14, wherein one of the first sensors is closest to a rotation axis of the drive shaft in a radial direction of the drive shaft, and another one of the first sensors is farthest from the rotation axis of the drive shaft in the radial direction.

16. The medical system according to claim 15, wherein the controller is configured to control the rollers not to move the drive shaft or change the orientation upon receipt of the signals indicating that said one of the first sensors is contacting the object or the biological tissue and indicating that said another one of the first sensor is not contacting the object or the biological tissue.

17. The medical system according to claim 13, wherein
the medical device further includes a second sensor disposed inside the distal end tube and configured to detect contact with the guide wire and output a signal indicating whether the second sensor is contacting the guide wire, and
the controller is configured to control the rollers to change the orientation upon receipt of the signal indicating that the second sensor is contacting the guide wire.

18. The medical system according to claim 11, wherein the medical device further includes:
an outer shaft surrounding the drive shaft and including a distal end portion that is in contact with or adjacent to the cutter, and
a third sensor attached to the distal end portion of the outer shaft and configured to detect contact with the object or the biological tissue and output a signal indicating whether the third sensor is contacting the object or the biological tissue.

19. The medical system according to claim 18, wherein
the medical device further includes a fourth sensor disposed on a proximal side of the distal end portion of the outer shaft and configured to detect contact with the object or the biological tissue and output a signal indicating whether the fourth sensor is contacting the object or the biological tissue, and
the controller is configured to control the rollers to move the drive shaft toward a distal direction or change the orientation upon receipt of the signal indicating that the fourth sensor is contacting the object or the biological tissue.

20. The medical system according to claim 11, wherein
the medical device further includes a guide portion that passes through the drive shaft and protrudes from the cutter toward a distal direction, and
the first sensor is disposed at a distal end of the guide portion.
